(19) United States
(12) Patent Application Publication
(10) Pub. No.: US 2025/0249235 A1
(43) Pub. Date: Aug. 7, 2025

(54) Deep Brain Stimulation Neuromodulation Targeting

(71) Applicant: Boston Scientific Neuromodulation Corporation, Valencia, CA (US)

(72) Inventors: Javad Paknahad, Pasadena, CA (US); Tianhe Zhang, Studio City, CA (US); G. Karl Steinke, Valencia, CA (US); Soroush Niketeghad, Saugus, CA (US)

(21) Appl. No.: 19/044,030

(22) Filed: Feb. 3, 2025

Related U.S. Application Data
(60) Provisional application No. 63/550,453, filed on Feb. 6, 2024.

(51) Int. Cl. A61N 1/05; A61N 1/36

(52) U.S. Cl. CPC A61N 1/0534; A61N 1/36139

(57) ABSTRACT

Methods and systems for providing deep brain stimulation (DBS) for a patient are described. Evoked potentials (EPs) evoked by the stimulation are recorded and compared to modeled EPs. The modeled EPs are determined based on an overlap of stimulation field models (SFMs) for a given set of stimulation parameters with a target volume of the patient's brain, the target region being the source of the EPs. The target volume may include the patient's subthalamic nucleus (STN), for example. The modeled EPs are used to predict electrical signals that will be sensed at recording electrodes of an electrode lead. The recorded EPs can be compared to the modeled electrical signals to guide aspects of the stimulation therapy.

### CROSS REFERENCE TO RELATED APPLICATIONS

This application is a non-provisional of U.S. Provisional Patent Application Ser. No. 63/550,453, filed Feb. 6, 2024, which is incorporated herein by reference in its entirety, and to which priority is claimed.

### FIELD OF THE INVENTION

This application relates to deep brain stimulation (DBS), and more particularly, to methods and systems for using sensed neural responses for facilitating aspects of DBS.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) context. DBS has been applied therapeutically for the treatment of neurological disorders, including Parkinson's Disease (PD), essential tremor, dystonia, and epilepsy, to name but a few. Further details discussing the treatment of diseases using DBS are disclosed in U.S. Pat. Nos. 6,845,267, and 6,950,707. However, the present invention may find applicability with any implantable neurostimulator device system.

Each of these neurostimulation systems, whether implantable or external, typically includes one or more electrode-carrying stimulation leads, which are implanted at the desired stimulation site, and a neurostimulator, used externally or implanted remotely from the stimulation site, but coupled either directly to the neurostimulation lead(s) or indirectly to the neurostimulation lead(s) via a lead extension. The neurostimulation system may further comprise a handheld external control device to remotely instruct the neurostimulator to generate electrical stimulation pulses in accordance with selected stimulation parameters. Typically, the stimulation parameters programmed into the neurostimulator can be adjusted by manipulating controls on the external control device to modify the electrical stimulation provided by the neurostimulator system to the patient.

Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated to provide the therapeutic benefit (e.g., treatment of movement disorders), while minimizing the volume of non-target tissue that is stimulated. A typical stimulation parameter set may include the electrodes that are acting as anodes or cathodes, as well as the amplitude, duration, and rate of the stimulation pulses.

Non-optimal electrode placement and stimulation parameter selections may result in excessive energy consumption due to stimulation that is set at too high amplitude, too wide a pulse duration, or too fast a frequency; inadequate or marginalized treatment due to stimulation that is set at too low an amplitude, too narrow a pulse duration, or too slow a frequency; or stimulation of neighboring cell populations that may result in undesirable side effects. For example, bilateral DBS of the subthalamic nucleus (STN) has been shown to provide effective therapy for improving the major motor signs of advanced Parkinson's disease, and although the bilateral stimulation of the subthalamic nucleus is considered safe, an emerging concern is the potential negative consequences that it may have on cognitive functioning and overall quality of life (see A. M. M. Frankemolle, et al., Reversing Cognitive-Motor Impairments in Parkinson's Disease Patients Using a Computational Modelling Approach to Deep Brain Stimulation Programming, Brain 2010; pp. 1-16). In large part, this phenomenon is due to the small size of the subthalamic nucleus. Even with the electrodes located predominately within the sensorimotor territory, the electrical field generated by DBS is non-discriminately applied to all neural elements surrounding the electrodes, thereby resulting in the spread of current to neural elements affecting cognition. As a result, diminished cognitive function during stimulation of the subthalamic nucleus may occur do to non-selective activation of non-motor pathways within or around the subthalamic nucleus.

The large number of electrodes available, combined with the ability to generate a variety of complex stimulation pulses, presents a huge selection of stimulation parameter sets to the clinician or patient. In the context of DBS, neurostimulation leads with a complex arrangement of electrodes that not only are distributed axially along the leads but are also distributed circumferentially around the neurostimulation leads as segmented electrodes, can be used.

To facilitate such selection, the clinician generally programs the external control device, and if applicable the neurostimulator, through a computerized programming system. This programming system can be a self-contained hardware/software system or can be defined predominantly by software running on a standard personal computer (PC) or mobile platform. The PC or custom hardware may actively control the characteristics of the electrical stimulation generated by the neurostimulator to allow the optimum stimulation parameters to be determined based on patient feedback and to subsequently program the external control device with the optimum stimulation parameters.

When electrical leads are implanted within the patient, the computerized programming system may be used to instruct the neurostimulator to apply electrical stimulation to test placement of the leads and/or electrodes, thereby assuring that the leads and/or electrodes are implanted in effective locations within the patient. The system may also instruct the user how to improve the positioning of the leads or confirm when a lead is well-positioned. Once the leads are correctly positioned, a fitting procedure, which may be referred to as a navigation session, may be performed using the computerized programming system to program the external control device, and if applicable the neurostimulator, with a set of stimulation parameters that best addresses the neurological disorder(s).

### SUMMARY

Disclosed herein is a method of providing deep brain stimulation (DBS) to a patient's brain using one or more electrode leads implanted in the patient's brain, wherein each of the one or more leads comprises a plurality of electrodes, the method comprising: using a first one or more of the electrodes to provide active stimulation to the patient's brain, using a second one or more of the electrodes to record one or more electrical signals indicative of evoked potentials (EPs) evoked by a target volume of the patient's brain, comparing the one or more recorded signals to a plurality of modeled EPs, wherein each of the modeled EPs comprise predicted electrical signals at one or more of the electrodes in response to activation of the target volume by modeled stimulation with a predefined set of model stimulation parameters, and using the comparison to adjust the stimulation. According to some embodiments, comparing the one or more recorded signal to the modeled EPs comprises extracting one or more features of the recorded signals and comparing the extracted features to corresponding features of the modeled EPs. According to some embodiments, the one or more extracted features comprise one or more of one or more peak heights, one or more peak-peak heights, areas under one or more peaks, one or more latencies, and one or more peak-peak ratios. According to some embodiments, comparing the one or more recorded signal to the modeled EPs comprises overlaying the recorded signals and the modeled EPs. According to some embodiments, the modeled EPs are comprised within a look-up table (LUT). According to some embodiments, adjusting the stimulation comprises adjusting the stimulation to provide stimulation to a larger portion of the target volume. According to some embodiments, adjusting the stimulation comprises adjusting one or more of an amplitude, pulse width, frequency, duty cycle, electrode position, or stimulation location. According to some embodiments, adjusting the stimulation comprises adjusting which electrodes are active for providing stimulation and/or adjusting a fractionation of current among the electrodes that are active. According to some embodiments, the method further comprises determining the modeled EPs for each of the predefined sets of model stimulation parameters. According to some embodiments, determining the modeled EPs comprises: for each of the predefined sets of model stimulation parameters, determining a stimulation field model (SFM) that predicts a volume of tissue activated using the set of model stimulation parameters, determining an overlap region of the SFM with the target volume, and predicting electrical signals that will be sensed at the one or more of the electrodes based on activation of neural elements within the overlap region. According to some embodiments, the method further comprises comparing the one or more recorded signals to the predicted electrical signals to predict an overlap of stimulation fields created by the active stimulation with the target volume. According to some embodiments, the target volume is determined using voxelized imaging data. According to some embodiments, the target volume comprises the patient's subthalamic nucleus (STN) or Globus Pallidus.

Also disclosed herein is a method of predicting evoked potentials (EPs) evoked in a patient's brain by electrical stimulation with one or more electrode leads implanted in the patient's brain, wherein each of the one or more leads comprises a plurality of electrodes, the method comprising: receiving a set of stimulation parameters, determining a stimulation field model (SFM) for the set of stimulation parameters, wherein the SFM predicts a volume of tissue activated using the set of stimulation parameters, determining an overlap region of the SFM with a target volume of the patient's brain, and predicting electrical potentials arising at one or more of the electrodes caused by activation of neural elements within the overlap region. According to some embodiments, the method further comprises determining the modeled EPs for each of the predefined sets of model stimulation parameters. According to some embodiments, determining the modeled EPs comprises: for each of the predefined sets of model stimulation parameters, determining a stimulation field model (SFM) that predicts a volume of tissue activated using the set of model stimulation parameters, determining an overlap region of the SFM with the target volume, and predicting electrical signals that will be sensed at the one or more of the electrodes based on activation of neural elements within the overlap region. According to some embodiments, the target volume is determined using voxelized imaging data. According to some embodiments, the target volume comprises the patient's subthalamic nucleus (STN).

Also disclosed herein is a system for providing deep brain stimulation (DBS) to a patient's brain using one or more electrode leads implanted in the patient's brain, wherein each of the one or more leads comprises a plurality of electrodes, the system comprising: control circuitry configured to execute a method comprising: using a first one or more of the electrodes to provide active stimulation to the patient's brain, using a second one or more of the electrodes to record one or more electrical signals indicative of evoked potentials (EPs) evoked by a target volume of the patient's brain, comparing the one or more recorded signals to a plurality of modeled EPs, wherein each of the modeled EPs comprise predicted electrical signals at one or more of the electrodes in response to activation of the target volume by modeled stimulation with a predefined set of model stimulation parameters, and using the comparison to adjust the stimulation. According to some embodiments, comparing the one or more recorded signal to the modeled EPs comprises extracting one or more features of the recorded signals and comparing the extracted features to corresponding features of the modeled EPs. According to some embodiments, the one or more extracted features comprise one or more of one or more peak heights, one or more peak-peak heights, areas under one or more peaks, one or more latencies, and one or more peak-peak ratios. According to some embodiments, comparing the one or more recorded signal to the modeled EPs comprises overlaying the recorded signals and the modeled EPs. According to some embodiments, the modeled EPs are comprised within a look-up table (LUT). According to some embodiments, adjusting the stimulation comprises adjusting the stimulation to provide stimulation to a larger portion of the target volume. According to some embodiments, adjusting the stimulation comprises adjusting one or more of an amplitude, pulse width, frequency, duty cycle, or stimulation location. According to some embodiments, adjusting the stimulation comprises adjusting which electrodes are active for providing stimulation and/or adjusting a fractionation of current among the electrodes that are active. According to some embodiments, the method further comprises determining the modeled EPs for each of the predefined sets of model stimulation parameters. According to some embodiments, determining the modeled EPs comprises: for each of the predefined sets of model stimulation parameters, determining a stimulation field model (SFM) that predicts a volume of tissue activated using the set of model stimulation parameters, determining an overlap region of the SFM with the target volume, predicting electrical signals that will be sensed at the one or more of the electrodes based on activation of neural elements within the overlap region. According to some embodiments, the method further comprises comparing the one or more recorded signals to the predicted electrical signals to predict an overlap of stimulation fields created by the active stimulation with the target volume. According to some embodiments, the target volume is determined using voxelized imaging data. According to some embodiments, the target volume comprises the patient's subthalamic nucleus (STN).

Also disclosed herein is a non-transitory computer readable medium for use in a system for providing deep brain stimulation (DBS) to a patient's brain using one or more electrode leads implanted in the patient's brain, wherein each of the one or more leads comprises a plurality of electrodes, the non-transitory computer readable medium comprising instructions, which when executed using a computer, cause the computer to execute a method comprising: using a first one or more of the electrodes to provide active stimulation to the patient's brain, using a second one or more of the electrodes to record one or more electrical signals indicative of evoked potentials (EPs) evoked by a target volume of the patient's brain, comparing the one or more recorded signals to a plurality of modeled EPs, wherein each of the modeled EPs comprise predicted electrical signals at one or more of the electrodes in response to activation of the target volume by modeled stimulation with a predefined set of model stimulation parameters, and using the comparison to adjust the stimulation. According to some embodiments, comparing the one or more recorded signal to the modeled EPs comprises extracting one or more features of the recorded signals and comparing the extracted features to corresponding features of the modeled EPs. According to some embodiments, the one or more extracted features comprise one or more of one or more peak heights, one or more peak-peak heights, areas under one or more peaks, one or more latencies, and one or more peak-peak ratios. According to some embodiments, comparing the one or more recorded signal to the modeled EPs comprises overlaying the recorded signals and the modeled EPs. According to some embodiments, the modeled EPs are comprised within a look-up table (LUT). According to some embodiments, adjusting the stimulation comprises adjusting the stimulation to provide stimulation to a larger portion of the target volume. According to some embodiments, adjusting the stimulation comprises adjusting one or more of an amplitude, pulse width, frequency, duty cycle, or stimulation location. According to some embodiments, adjusting the stimulation comprises adjusting which electrodes are active for providing stimulation and/or adjusting a fractionation of current among the electrodes that are active. According to some embodiments, the method further comprises determining the modeled EPs for each of the predefined sets of model stimulation parameters. According to some embodiments, determining the modeled EPs comprises: for each of the predefined sets of model stimulation parameters, determining a stimulation field model (SFM) that predicts a volume of tissue activated using the set of model stimulation parameters, determining an overlap region of the SFM with the target volume, predicting electrical signals that will be sensed at the one or more of the electrodes based on activation of neural elements within the overlap region. According to some embodiments, the method further comprises comparing the one or more recorded signals to the predicted electrical signals to predict an overlap of stimulation fields created by the active stimulation with the target volume. According to some embodiments, the target volume is determined using voxelized imaging data. According to some embodiments, the target volume comprises the patient's subthalamic nucleus (STN).

Also disclosed herein is a system for predicting evoked potentials (EPs) evoked in a patient's brain by electrical stimulation with one or more electrode leads implanted in the patient's brain, wherein each of the one or more leads comprises a plurality of electrodes, the system comprising: control circuitry configured to execute a method comprising: receiving a set of stimulation parameters, determining a stimulation field model (SFM) for the set of stimulation parameters, wherein the SFM predicts a volume of tissue activated using the set of stimulation parameters, determining an overlap region of the SFM with a target volume of the patient's brain, and predicting electrical potentials arising at one or more of the electrodes caused by activation of neural elements within the overlap region. According to some embodiments, the method further comprising determining the modeled EPs for each of the predefined sets of model stimulation parameters. According to some embodiments, determining the modeled EPs comprises: for each of the predefined sets of model stimulation parameters, determining a stimulation field model (SFM) that predicts a volume of tissue activated using the set of model stimulation parameters, determining an overlap region of the SFM with the target volume, and predicting electrical signals that will be sensed at the one or more of the electrodes based on activation of neural elements within the overlap region. According to some embodiments, the target volume is determined using voxelized imaging data. According to some embodiments, the target volume comprises the patient's subthalamic nucleus (STN).

The invention may also reside in the form of a programed external device (via its control circuitry) for carrying out the above methods, a programmed implantable pulse generator (IPG) or external trial stimulator (ETS) (via its control circuitry) for carrying out the above methods, a system including a programmed external device and IPG or ETS for carrying out the above methods, or as a computer-readable media for carrying out the above methods stored in an external device or IPG or ETS. The invention may also reside in one or more non-transitory computer-readable media comprising instructions, which when executed by a processor of a machine configure the machine to perform any of the above methods.

### BRIEF DESCRIPTION OF THE DRAWINGS

FIGS. 1A and 1B show an Implantable Pulse Generator (IPG) and electrode lead having split-ring electrodes, respectively.

FIGS. 2A and 2B show an example of stimulation pulses (waveforms) producible by the IPG or by an External Trial Stimulator (ETS).

FIG. 3 shows an example of stimulation circuitry useable in the IPG or ETS.

FIG. 4 shows an ETS environment used to provide stimulation before implantation of an IPG.

FIG. 5 shows various external devices capable of communicating with and programming stimulation in an IPG or ETS.

FIG. 6 illustrates sensing circuitry useable in an IPG.

FIG. 7 illustrates an embodiment of a user interface (UI) for programming stimulation.

FIGS. 8A and 8B illustrate evoked resonant neural activity (ERNA).

FIG. 9 illustrates an embodiment of a workflow for using modeled evoked potentials (EPs) to guide aspects of stimulation therapy.

FIG. 10 illustrates aspects of modeling electrical signals arising from EPs for a given set of stimulation parameters.

FIG. 11 illustrates an example of modeled EPs predicted at various electrode contacts for different stimulation parameters.

FIG. 12 illustrates modeled EP amplitudes as a function of stimulation amplitude.

### DETAILED DESCRIPTION

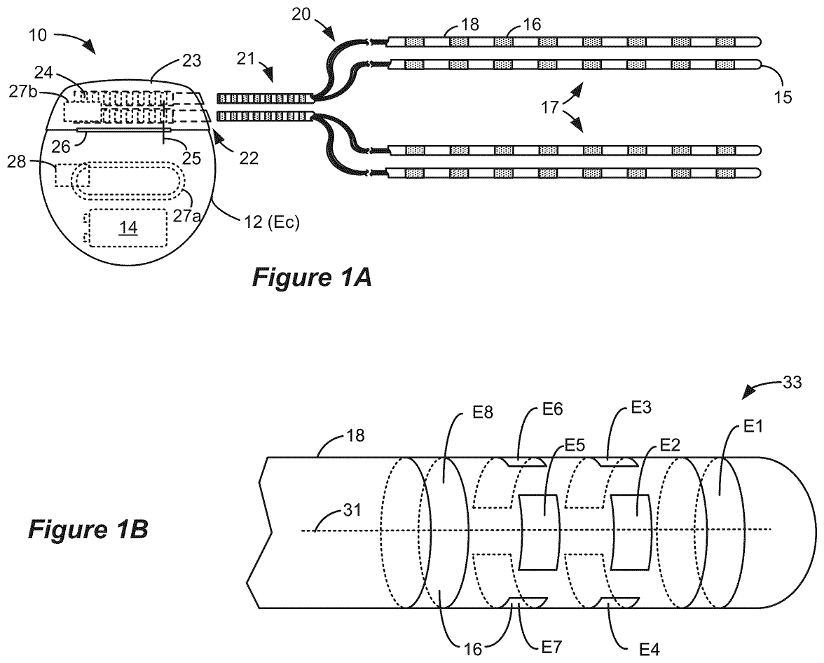

A DBS or SCS system typically includes an Implantable Pulse Generator (IPG) 10 shown in FIG. 1A. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more electrode leads 15 can be used having ring-shaped electrodes 16 carried on a flexible body 18.

In yet another example shown in FIG. 1B, an electrode lead 33 can include one or more split-ring electrodes. In this example, eight electrodes 16 (E1-E8) are shown. Electrode E1 at the distal end of the lead and electrode E8 at a proximal end of the lead comprise ring electrodes spanning 360 degrees around a central axis of the lead 33. In some embodiments, the electrode E1 may be a “bullet tip” electrode, meaning that it can cover the tip of the electrode lead. Electrodes E2, E3, and E4 comprise split-ring electrodes, each of which are located at the same longitudinal position along the central axis 31, but with each spanning less than 360 degrees around the axis. For example, each of electrodes E2, E3, and E4 may span 90 degrees around the axis 31, with each being separated from the others by gaps of 30 degrees. Electrodes E5, E6, and E7 also comprise split-ring electrodes, but are located at a different longitudinal position along the central axis 31 than are split ring electrodes E4, E2, and E3. As shown, the split-ring electrodes E2-E4 and E5-E7 may be located at longitudinal positions along the axis 31 between ring electrodes E1 and E8. However, this is just one example of a lead 33 having split-ring electrodes. In other designs, all electrodes can be split-ring, or there could be different numbers of split-ring electrodes at each longitudinal position (i.e., more or less than three), or the ring and split-ring electrodes could occur at different or random longitudinal positions, etc.

Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are thirty-two electrodes (E1-E32), split between four percutaneous leads 15, and thus the header 23 may include a 2×2 array of eight-electrode lead connectors 22. However, the type and number of leads, and the number of electrodes, in an IPG is application-specific and therefore can vary. The conductive case 12 can also comprise an electrode (Ec).

In a DBS application, as is useful in the treatment of tremor in Parkinson's disease for example, the IPG 10 is typically implanted under the patient's clavicle (collarbone). Lead wires 20 are tunneled through the neck and the scalp and the electrode leads 15 (or 33) are implanted through holes drilled in the skull and positioned for example in the subthalamic nucleus (STN) and the pedunculopontine nucleus (PPN) in each brain hemisphere.

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Bluetooth Low Energy (BLE), as described in U.S. Patent Publication 2019/0209851, Zigbee, WiFi, MICS, and the like.

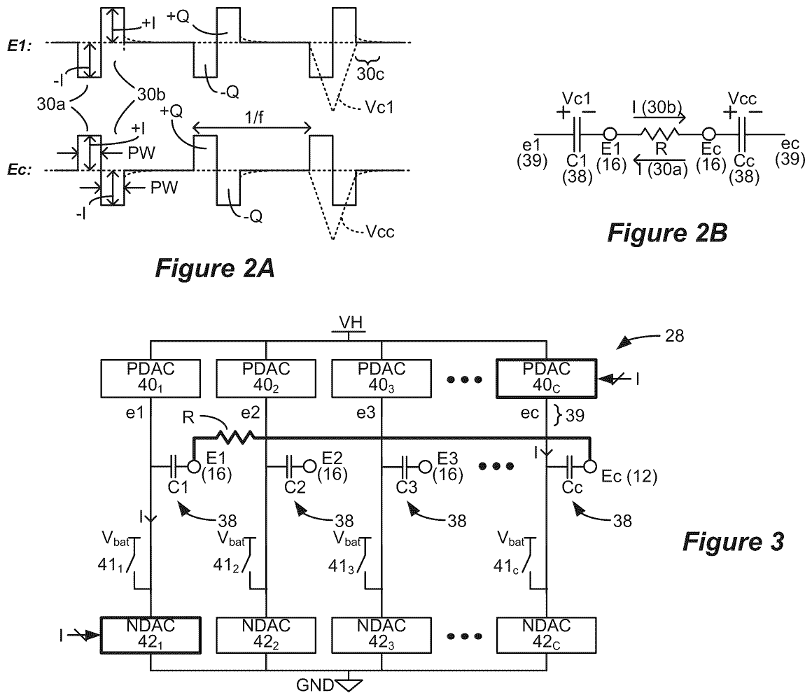

Stimulation in IPG 10 is typically provided by pulses each of which may include a number of phases such as 30a and 30b, as shown in the example of FIG. 2A. In the example shown, such stimulation is monopolar, meaning that a current is provided between at least one selected lead-based electrode (e.g., E1) and the case electrode Ec 12. Stimulation parameters typically include amplitude (current I, although a voltage amplitude V can also be used); frequency (f); pulse width (PW) of the pulses or of its individual phases such as 30a and 30b; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude +I to the tissue. Note that at any time the current sunk from the tissue (e.g., −I at E1 during phase 30a) equals the current sourced to the tissue (e.g., +I at Ec during phase 30a) to ensure that the net current injected into the tissue is zero. The polarity of the currents at these electrodes can be changed: Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40ᵢ and one or more current sinks 42ᵢ. The sources and sinks 40ᵢ and 42ᵢ can comprise Digital-to-Analog converters (DACs), and may be referred to as PDACs 40ᵢ and NDACs 42ᵢ in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node Ei 39 is connected to an electrode Ei 16 via a DC-blocking capacitor Ci 38, for the reasons explained below. PDACs 40ᵢ and NDACs 42ᵢ can also comprise voltage sources.

Proper control of the PDACs 40ᵢ and NDACs 42ᵢ allows any of the electrodes 16 and the case electrode Ec 12 to act as anodes or cathodes to create a current through a patient's tissue, R, hopefully with good therapeutic effect. In the example shown, and consistent with the first pulse phase 30a of FIG. 2A, electrode E1 has been selected as a cathode electrode to sink current from the tissue R and case electrode Ec has been selected as an anode electrode to source current to the tissue R. Thus, PDAC 40_{C} and NDAC 42₁ are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40ᵢ and the electrode nodes ei 39, and between the one or more NDACs 42ᵢ and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796. The stimulation circuitries described herein provide multiple independent current control (MICC) (or multiple independent voltage control) to guide the estimate of current fractionalization among multiple electrodes and estimate a total amplitude that provide a desired strength. In other words, the total anodic (or cathodic) current can be split among two or more electrodes and/or the total cathodic current can be split among two or more electrodes, allowing the stimulation location and resulting field shapes to be adjusted. For example, a “virtual electrode” may be created at a position between two physical electrodes by fractionating current between the two electrodes.

Much of the stimulation circuitry 28 of FIG. 3, including the PDACs 40ᵢ and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Eᵢ 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will build up across the DC-blocking capacitors C1 and Cc associated with the electrodes E1 and Ec used to produce the current, giving rise to voltages Vc1 and Vcc which decrease in accordance with the amplitude of the current and the capacitance of the capacitors 38 (dV/dt=I/C). During the second pulse phase 30b, when the polarity of the current I is reversed at the selected electrodes E1 and Ec, the stored charge on capacitors C1 and Cc is actively recovered, and thus voltages Vc1 and Vcc increase and return to 0V at the end of the second pulse phase 30b.

To recover all charge by the end of the second pulse phase 30b of each pulse (Vc1=Vcc=0V), the first and second phases 30a and 30b are charged balanced at each electrode, with the first pulse phase 30a providing a charge of −Q (−I*PW) and the second pulse phase 30b providing a charge of +Q (+I*PW) at electrode E1, and with the first pulse phase 30a providing a charge of +Q and the second pulse phase 30b providing a charge of −Q at the case electrode Ec. In the example shown, such charge balancing is achieved by using the same pulse width (PW) and the same amplitude (|I|) for each of the opposite-polarity pulse phases 30a and 30b. However, the pulse phases 30a and 30b may also be charged balanced at each electrode if the product of the amplitude and pulse widths of the two phases 30a and 30b are equal, or if the area under each of the phases is equal, as is known.

FIG. 3 shows that stimulation circuitry 28 can include passive recovery switches 41ᵢ, which are described further in U.S. Patent Application Publications 2018/0071527 and 2018/0140831. Passive recovery switches 41; may be attached to each of the electrode nodes ei 39, and are used to passively recover any charge remaining on the DC-blocking capacitors Ci 38 after issuance of the second pulse phase 30b—i.e., to recover charge without actively driving a current using the DAC circuitry. Passive charge recovery can be prudent, because non-idealities in the stimulation circuitry 28 may lead to pulse phases 30a and 30b that are not perfectly charge balanced.

Therefore, and as shown in FIG. 2A, passive charge recovery typically occurs after the issuance of second pulse phases 30b, for example during at least a portion 30c of the quiet periods between the pulses, by closing passive recovery switches 41ᵢ. As shown in FIG. 3, the other end of the switches 41ᵢ not coupled to the electrode nodes ei 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 by placing the capacitors in parallel between the reference voltage (Vbat) and the patient's tissue. Note that passive charge recovery is illustrated as small exponentially decaying curves during 30c in FIG. 2A, which may be positive or negative depending on whether pulse phase 30a or 30b have a predominance of charge at a given electrode.

Passive charge recovery 30c may alleviate the need to use biphasic pulses for charge recovery, especially in the DBS context when the amplitudes of currents may be lower, and therefore charge recovery is less of a concern. For example, and although not shown in FIG. 2A, the pulses provided to the tissue may be monophasic, comprising only a first pulse phase 30a. This may be followed thereafter by passive charge recovery 30c to eliminate any charge build up that occurred during the singular pulses 30a.

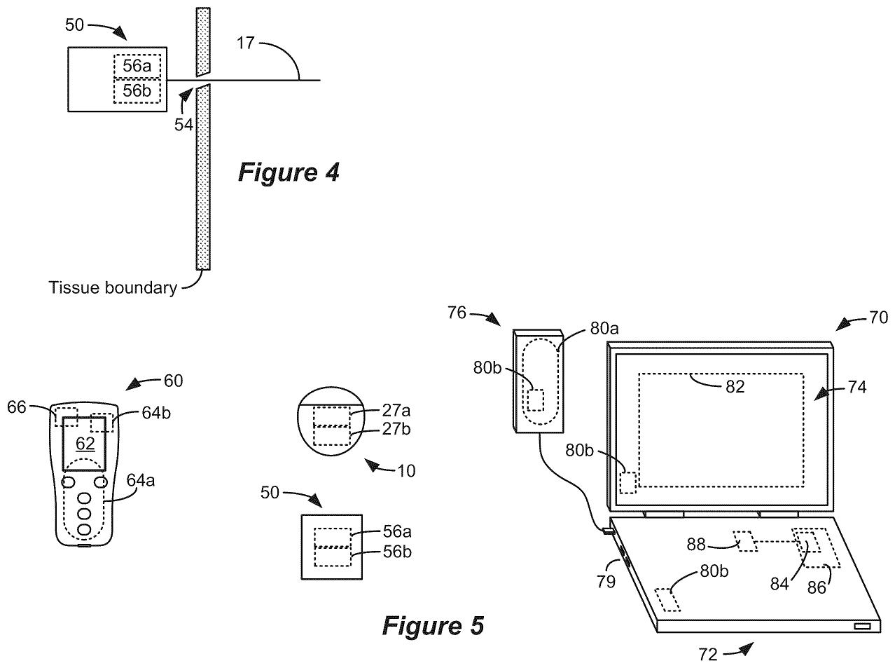

FIG. 4 shows an external trial stimulation environment that may precede implantation of an IPG 10 in a patient, for example, during the operating room to test stimulation and confirm the lead position. During external trial stimulation, stimulation can be tried on the implant patient to evaluate side-effect thresholds and confirm that the lead is not too close to structures that cause side effects. Like the IPG 10, the external trial stimulator (ETS) 50 can include one or more antennas to enable bi-directional communications with external devices such as those shown in FIG. 5. Such antennas can include a near-field magnetic-induction coil antenna 56a, and/or a far-field RF antenna 56b, as described earlier. ETS 50 may also include stimulation circuitry able to form stimulation in accordance with a stimulation program, which circuitry may be similar to or comprise the same stimulation circuitry 28 (FIG. 3) present in the IPG 10. ETS 50 may also include a battery (not shown) for operational power. The sensing capabilities described herein with regard to the IPG 10, may also be included in the ETS 50 for the purposes described below. As the IPG may include a case electrode, an ETS may provide one or more connections to establish similar returns; for example, using patch electrodes. Likewise, the ETS may communicate with the clinician programmer (CP) so that the CP can process the data as described below.

FIG. 5 shows various external devices that can wirelessly communicate data with the IPG 10 or ETS 50, including a patient hand-held external controller 60, and a clinician programmer (CP) 70. Both of devices 60 and 70 can be used to wirelessly transmit a stimulation program to the IPG 10 or ETS 50—that is, to program their stimulation circuitries to produce stimulation with a desired amplitude and timing described earlier. Both devices 60 and 70 may also be used to adjust one or more stimulation parameters of a stimulation program that the IPG 10 is currently executing. Devices 60 and 70 may also wirelessly receive information from the IPG 10 or ETS 50, such as various status information, etc.

External controller 60 can be as described in U.S. Patent Application Publication 2015/0080982 for example and may comprise a controller dedicated to work with the IPG 10 or ETS 50. External controller 60 may also comprise a general-purpose mobile electronics device such as a mobile phone which has been programmed with a Medical Device Application (MDA) allowing it to work as a wireless controller for the IPG 10 or ETS, as described in U.S. Patent Application Publication 2015/0231402. External controller 60 includes a user interface, preferably including means for entering commands (e.g., buttons or selectable graphical elements) and a display 62. The external controller 60's user interface enables a patient to adjust stimulation parameters, although it may have limited functionality when compared to the more-powerful clinician programmer 70, described shortly.

The external controller 60 can have one or more antennas capable of communicating with the IPG 10. For example, the external controller 60 can have a near-field magnetic-induction coil antenna 64a capable of wirelessly communicating with the coil antenna 27a or 56a in the IPG 10 or ETS 50. The external controller 60 can also have a far-field RF antenna 64b capable of wirelessly communicating with the RF antenna 27b or 56b in the IPG 10 or ETS 50.

Clinician programmer 70 is described further in U.S. Patent Application Publication 2015/0360038, and can comprise a computing device 72, such as a desktop, laptop, or notebook computer, a tablet, a mobile smart phone, a Personal Data Assistant (PDA)-type mobile computing device, etc. In FIG. 5, computing device 72 is shown as a laptop computer that includes typical computer user interface means such as a screen 74, a mouse, a keyboard, speakers, a stylus, a printer, etc., not all of which are shown for convenience. Also shown in FIG. 5 are accessory devices for the clinician programmer 70 that are usually specific to its operation as a stimulation controller, such as a communication “wand” 76 coupleable to suitable ports on the computing device 72, such as USB ports 79 for example.

The antenna used in the clinician programmer 70 to communicate with the IPG 10 or ETS 50 can depend on the type of antennas included in those devices. If the patient's IPG 10 or ETS 50 includes a coil antenna 27a or 56a, wand 76 can likewise include a coil antenna 80a to establish near-field magnetic-induction communications at small distances. In this instance, the wand 76 may be affixed in close proximity to the patient, such as by placing the wand 76 in a belt or holster wearable by the patient and proximate to the patient's IPG 10 or ETS 50. If the IPG 10 or ETS 50 includes an RF antenna 27b or 56b, the wand 76, the computing device 72, or both, can likewise include an RF antenna 80b to establish communication at larger distances. The clinician programmer 70 can also communicate with other devices and networks, such as the Internet, either wirelessly or via a wired link provided at an Ethernet or network port.

To program stimulation programs or parameters for the IPG 10 or ETS 50, the clinician interfaces with a clinician programmer graphical user interface (GUI) 82 provided on the display 74 of the computing device 72. As one skilled in the art understands, the GUI 82 can be rendered by execution of clinician programmer software 84 stored in the computing device 72, which software may be stored in the device's non-volatile memory 86. Execution of the clinician programmer software 84 in the computing device 72 can be facilitated by control circuitry 88 such as one or more microprocessors, microcomputers, FPGAs, DSPs, other digital logic structures, etc., which are capable of executing programs in a computing device, and which may comprise their own memories. For example, control circuitry 88 can comprise an i5 processor manufactured by Intel Corp, as described at https://www.intel.com/content/www/us/en/products/processors/core/i5-processors.html. Such control circuitry 88, in addition to executing the clinician programmer software 84 and rendering the GUI 82, can also enable communications via antennas 80a or 80b to communicate stimulation parameters chosen through the GUI 82 to the patient's IPG 10.

The user interface of the external controller 60 may provide similar functionality because the external controller 60 can include similar hardware and software programming as the clinician programmer. For example, the external controller 60 includes control circuitry 66 similar to the control circuitry 88 in the clinician programmer 70 and may similarly be programmed with external controller software stored in device memory.

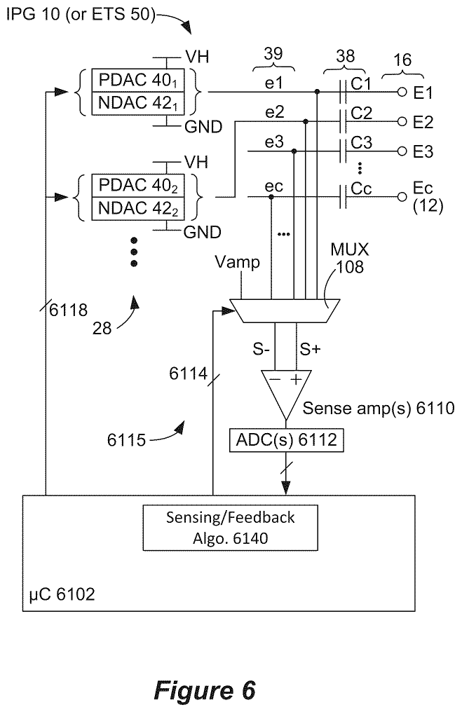

An increasingly interesting development in pulse generator systems is the addition of sensing capability to complement the stimulation that such systems provide. FIG. 6 shows an IPG 10 that includes stimulation and sensing functionality. (An ETS as described earlier could also include stimulation and sensing capabilities). FIG. 6 shows further details of the circuitry in an IPG 10 (and/or ETS) that can provide stimulation and sensing innate or evoked signals. The IPG 10 includes control circuitry 6102, which may comprise a microcontroller, such as Part Number MSP430, manufactured by Texas Instruments, Inc., which is described in data sheets at http://www.ti.com/microcontrollers/msp430-ultra-low-power-mcus/overview.html, which are incorporated herein by reference. Other types of controller circuitry may be used in lieu of a microcontroller as well, such as microprocessors, FPGAs, DSPs, or combinations of these, etc. Control circuitry 6102 may also be formed in whole or in part in one or more Application Specific Integrated Circuits (ASICs), such as those described and incorporated earlier. The control circuitry 102 may be configured with one or more sensing/feedback algorithms 6140 that are configured to cause the IPG/ETS to sense neural signals and make certain adjustments and/or take certain actions based on the sensed neural signals. The sensing/feedback control algorithms may be configured within memory of the IPG.

The IPG 10 also includes stimulation circuitry 28 to produce stimulation at the electrodes 16, which may comprise the stimulation circuitry 28 shown earlier (FIG. 3). A bus 6118 provides digital control signals from the control circuitry 6102 to one or more PDACs 40ᵢ or NDACs 42ᵢ to produce currents or voltages of prescribed amplitudes (I) for the stimulation pulses, and with the correct timing (PW, F) at selected electrodes. As noted earlier, the DACs can be powered between a compliance voltage VH and ground. As also noted earlier, but not shown in FIG. 4, switch matrices could intervene between the PDACs and the electrode nodes 39, and between the NDACs and the electrode nodes 39, to route their outputs to one or more of the electrodes, including the conductive case electrode 12 (Ec). Control signals for switch matrices, if present, may also be carried by bus 118. Notice that the current paths to the electrodes 16 include the DC-blocking capacitors 38 described earlier, which provide safety by preventing the inadvertent supply of DC current to an electrode and to a patient's tissue. Passive recovery switches 41ᵢ (FIG. 3) could also be present but are not shown in FIG. 6 for simplicity.

IPG 10 also includes sensing circuitry 6115, and one or more of the electrodes 16 can be used to sense innate or evoked electrical signals, e.g., biopotentials from the patient's tissue. In this regard, each electrode node 39 can further be coupled to a sense amp circuit 6110. Under control by bus 6114, a multiplexer 6108 can select one or more electrodes to operate as sensing electrodes (S+, S−) by coupling the electrode(s) to the sense amps circuit 6110 at a given time, as explained further below. Although only one multiplexer 6108 and sense amp circuit 6110 are shown in FIG. 6, there could be more than one. For example, there can be four multiplexer 6108/sense amp circuit 6110 pairs each operable within one of four timing channels supported by the IPG 10 to provide stimulation. The sensed signals output by the sense amp circuitry are preferably converted to digital signals by one or more Analog-to-Digital converters (ADC(s)) 6112, which may sample the output of the sense amp circuit 6110 at 50 kHz for example. The ADC(s) 6112 may also reside within the control circuitry 6102, particularly if the control circuitry 6102 has A/D inputs. Multiplexer 6108 can also provide a fixed reference voltage, Vamp, to the sense amp circuit 6110, as is useful in a single-ended sensing mode (i.e., to set S− to Vamp).

So as not to bypass the safety provided by the DC-blocking capacitors 38, the inputs to the sense amp circuitry 110 are preferably taken from the electrode nodes 39. However, the DC-blocking capacitors 38 will pass AC signal components (while blocking DC components), and thus AC components within the signals being sensed will still readily be sensed by the sense amp circuitry 6110. In other examples, signals may be sensed directly at the electrodes 16 without passage through intervening capacitors 38.

According to some embodiments, it may be preferred to sense signals differentially, and in this regard, the sense amp circuitry 6110 comprises a differential amplifier receiving the sensed signal S+ (e.g., E3) at its non-inverting input and the sensing reference S− (e.g., E1) at its inverting input. As one skilled in the art understands, the differential amplifier will subtract S− from S+ at its output, and so will cancel out any common mode voltage from both inputs. This can be useful for example when sensing various neural signals, as it may be useful to subtract the relatively large-scale stimulation artifact from the measurement (as much as possible).

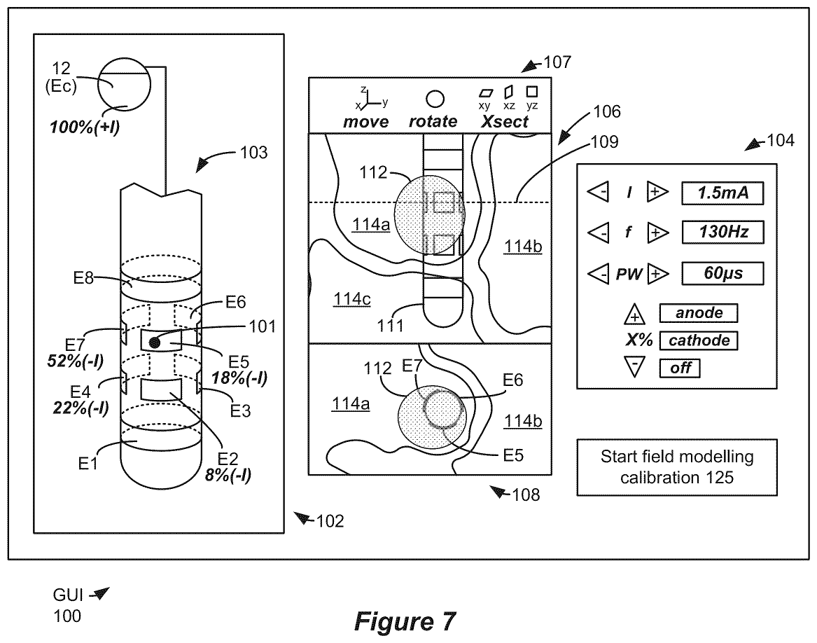

Particularly in the DBS context, it can be useful to provide a clinician with a visual indication of how stimulation selected for a patient will interact with the tissue in which the electrodes are implanted. This is illustrated in FIG. 7, which shows a Graphical User Interface (GUI) 100 operable on an external device capable of communicating with an IPG 10 or ETS 50. Typically, and as assumed in the description that follows, GUI 100 would be rendered on a clinician programmer 70 (FIG. 5), which may be used during surgical implantation of the leads to inform lead placement, or after implantation when a therapeutically useful stimulation program is being chosen for a patient. However, GUI 100 could be rendered on a patient external programmer 60 (FIG. 5) or any other external device capable of communicating with the IPG 10 or ETS 50.

GUI 100 allows a clinician (or patient) to select the stimulation program that the IPG 110 or ETS 50 will provide and provides options that control sensing of innate or evoked responses, as described below. In this regard, the GUI 100 may include a stimulation parameter interface 104 where various aspects of the stimulation program can be selected or adjusted. For example, interface 104 allows a user to select the amplitude (e.g., a current I) for stimulation; the frequency (f) of stimulation pulses; and the pulse width (PW) of the stimulation pulses. Stimulation parameter interface 104 can be significantly more complicated, particularly if the IPG 10 or ETS 50 supports the provision of stimulation that is more complicated than a repeating sequence of pulses. See, e.g., U.S. Patent Application Publication 2018/0071513. Nonetheless, interface 104 is simply shown for simplicity in FIG. 7 as allowing only for amplitude, frequency, and pulse width adjustment. Stimulation parameter interface 104 may include inputs to allow a user to select whether stimulation will be provided using biphasic (FIG. 2A) or monophasic pulses, and to select whether passive charge recovery will be used, although again these details aren't shown for simplicity.

Stimulation parameter interface 104 may further allow a user to select the active electrodes—i.e., the electrodes that will receive the prescribed pulses. Selection of the active electrodes can occur in conjunction with a leads interface 102, which can include an image 103 of the one or more leads that have been implanted in the patient. Although not shown, the leads interface 102 can include a selection to access a library of relevant images 103 of the types of leads that may be implanted in different patients.

In the example shown in FIG. 7, the leads interface 102 shows an image 103 of a single split-ring lead 33 like that described earlier with respect to FIG. 1B. The leads interface 102 can include a cursor 101 that the user can move (e.g., using a mouse connected to the clinician programmer 70) to select an illustrated electrode 16 (e.g., E1-E8, or the case electrode Ec). Once an electrode has been selected, the stimulation parameter interface 104 can be used to designate the selected electrode as an anode that will source current to the tissue, or as a cathode that will sink current from the tissue. Further, the stimulation parameter interface 104 allows the amount of the total anodic or cathodic current +I or −I that each selected electrode will receive to be specified in terms of a percentage, X. For example, in FIG. 7, the case electrode 12 Ec is specified to receive X=100% of the current I as an anodic current +I. The corresponding cathodic current −I is split between electrodes E5 (0.18*−I), E7 (0.52*−I), E2 (0.08*−I), and E4 (0.22*−I). Thus, two or more electrodes can be chosen to act as anodes or cathodes at a given time using MICC (as described above), allowing the electric field in the tissue to be shaped. The currents specified at the selected electrodes can be those provided during a first pulse phase (if biphasic pulses are used), or during an only pulse phase (if monophasic pulses are used).

GUI 100 can further include a visualization interface 106 that can allow a user to view an indication of the effects of stimulation, such as a stimulation field model (SFM) 112 (also referred to as a volume of tissue activated (VTA)) formed using the selected stimulation parameters. The SFM 112 is formed by field modelling, for example, in the clinician programmer 70. The illustrated embodiment of the GUI 99 includes a selection option 125 for initiating such modeling. Only one lead is shown in the visualization interface 106 for simplicity, although again a given patient might be implanted with more than one lead. Visualization interface 106 provides an image 111 of the lead(s) which may be three-dimensional.

The visualization interface 106 preferably, but not necessarily, further includes tissue imaging information 114 taken from the patient, represented as three different tissue structures 114a, 114b and 114c in FIG. 7 for the patient in question, which tissue structures may comprise different areas of the brain for example. Such tissue imaging information may comprise a Magnetic Resonance Image (MRI), a Computed Tomography (CT) image or other type of image. Often, one or more images, such as an MRI, CT, and/or a brain atlas are scaled and combined in a single image model. This allows the clinician programmer 70 on which GUI 100 is rendered to overlay the lead image 111 and the SFM 112 with the tissue imaging information in the visualization interface 106 so that the position of the SFM 112 relative to the various tissue structures 114i can be visualized. The image of the patient's tissue may also be taken after implantation of the lead(s), or tissue imaging information may comprise a generic image pulled from a library which is not specific to the patient in question, in some embodiments.

The various images shown in the visualization interface 106 (i.e., the lead image 111, the SFM 112, and the tissue structures 114i) can be three-dimensional in nature, and hence may be rendered in the visualization interface 106 in a manner to allow such three-dimensionality to be better appreciated by the user, such as by shading or coloring the images, etc. Additionally, a view adjustment interface 107 may allow the user to move or rotate the images, using cursor 101 for example.

GUI 100 can further include a cross-section interface 108 to allow the various images to be seen in a two-dimensional cross section. Specifically, cross-section interface 108 shows a particular cross section 109 taken perpendicularly to the lead image 111 and through split-ring electrodes E5, E6, and E7. This cross section 109 can also be shown in the visualization interface 106, and the view adjustment interface 107 can include controls to allow the user to specify the plane of the cross section 109 (e.g., in XY, XZ, or YZ planes) and to move its location in the image. Once the location and orientation of the cross section 109 is defined, the cross-section interface 108 can show additional details. For example, the SFM 112 can allow the user to get a sense of the strength and reach of the stimulation at different locations. Although GUI 100 includes stimulation definition (102, 104) and imaging (108, 106) in a single screen of the GUI, these aspects can also be separated as part of the GUI 100 and made accessible through various menu selections, etc.

Especially in a DBS application, it is important that correct stimulation parameters be determined for a given patient. Improper stimulation parameters may not yield effective relief of a patient's symptoms or may cause unwanted side effects. To determine proper stimulation, a clinician typically uses a GUI such as GUI 100 to try different combinations of stimulation parameters. This may occur, at least in part, during a DBS patient's surgery when the leads are being implanted. Such intra-operative determination of stimulation parameters can be useful to determine a general efficacy of DBS therapy. However, finalizing stimulation parameters that are appropriate for a given DBS patient typically occurs after surgery after the patient has had a chance to heal, and after the position of the leads stabilize in the patient. Thus, the patient will typically present to the clinician's office to determine (or further refine) optimal stimulation parameters during a programming session, often referred to as a “fitting session.”

Gauging the effectiveness of a given set of stimulation parameters typically involves programming the IPG 10 with that set, and then reviewing the therapeutic effectiveness and side effects that result. Therapeutic effectiveness and side effects are often assessed by one or more different scores (S) for one or more different clinical responses, which are entered into the GUI 99 of the clinician programmer 70 where they are stored with the stimulation parameters set being assessed. Such scores can be subjective in nature, based on patient or clinician observations. For example, bradykinesia (slowness of movement), rigidity, tremor, or other symptoms or side effects, can be scored by the patient, or by the clinician upon observing or questioning the patient. Such scores in one example can range from 0 (best) to 4 (worst). Scores can also be objective in nature based on measurements taken regarding a patient's symptoms or side effects. For example, a Parkinson's patient may be fitted with a wearable sensor that measures tremors, such as by measuring the frequency and amplitude of such tremors. A wearable sensor may communicate such metrics back to the GUI 99, and if necessary, converted to a score.

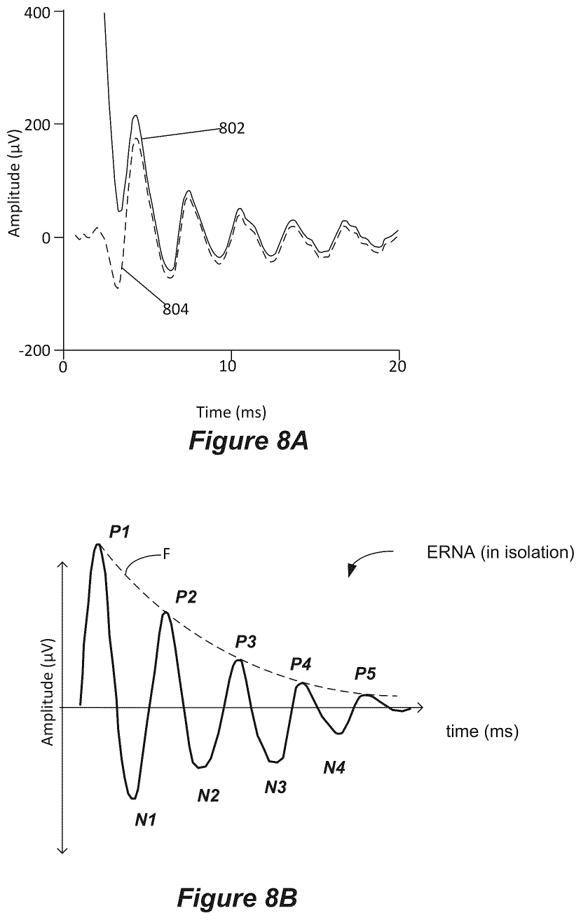

It has been observed that DBS stimulation in certain positions in the brain can evoke neural responses, i.e., electrical activity from neural elements, which may be measured. One example of such neural responses are resonant neural responses, referred to herein as evoked resonant neural activity (ERNAs). See, e.g., Sinclair, et al., “Subthalamic Nucleus Deep Brain Stimulation Evokes Resonant Neural Activity,” Ann. Neurol. 83(5), 1027-31, 2018. The ERNA responses typically have an oscillation frequency of about 200 to about 500 Hz and amplitudes of about 20 to about 200 μV. Stimulation of the STN, and particularly of the dorsal subregion of the STN, has been observed to evoke strong ERNA responses, whereas stimulation of the posterior subthalamic area (PSA) does not evoke such responses. Thus, ERNA can provide a biomarker for electrode location, which can potentially indicate acceptable or perhaps optimal lead placement and/or stimulation field placement for achieving the desired therapeutic response. FIG. 8A illustrates an example of an ERNA epoch after filtering 802 and after down-sampling 804 and removal of the residual stimulation artifact. FIG. 8B illustrates an example of an idealized ERNA in isolation. The ERNA comprises several positive peaks Pₙ and negative peaks Nₙ, which may have one or more characteristic amplitudes, lengths, separations, latencies, or other features. The ERNA signal may decay according to a characteristic decay function F. Such oscillatory evoked neural responses may also be referred to as DBS Local Evoked Potentials (DLEPs) and/or Evoked Oscillating Neural Responses (EONRs). The term ERNA will be used in this disclosure to refer to oscillatory evoked neural potentials in the patient's brain that are synchronized with stimulation, such as those illustrated in FIGS. 8A and 8B. It will be appreciated that the term ERNA refers to such signals, whether or not the signals are “resonant” in the strictest mathematical or physiological sense. More generally, such evoked neural responses in the patient's brain may simply be referred to as evoked neural responses and/or evoked potentials (EPs).

This disclosure particularly relates to methods, workflows, and systems for using recorded EPs, as a biomarker to inform aspects of neuromodulation therapy, such as DBS therapy. According to embodiments of the disclosure, recorded/sensed EP signals can provide a biomarker indicative that appropriate neural structures for addressing the patient's symptoms are being stimulated. For example, the dorsal region of the STN is a target for stimulation for the treatment of PD. Stimulation of that region has been shown to result in strong EPs. Thus, EPs can provide a biomarker that indicates that stimulation is effectively modulating the targeted dorsal region of the STN. Aspects of the disclosed methods and system involve biophysical models that predict electrical signals that are sensed at the electrodes of the electrode lead(s) when a target neural structure or neural volume (e.g., the dorsal region of the STN) is stimulated.

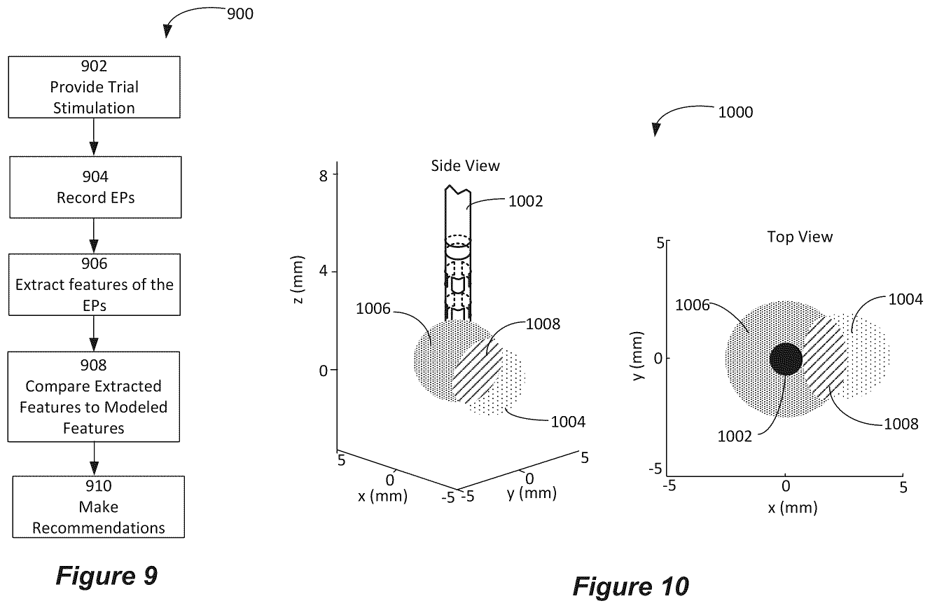

FIG. 9 illustrates an embodiment of a workflow 900 according to aspects of the disclosure. The workflow 900 may be used to determine the best stimulation parameters for a patient having one or more electrode leads implanted in their brain. For example, the workflow may help the clinician determine the best pulse width, stimulation amplitude, frequency, electrode configuration (i.e., which electrodes are used to deliver current and how the current is fractioned among the electrodes), etc. Specifically, in that embodiment, the goal is to determine stimulation parameters that generate a stimulation field that best overlaps with a target volume of the patient's brain and thereby modulates the neural elements within that target volume. Likewise, embodiments of the workflow may be used during the implantation procedure to inform the positioning of the electrode leads in the patient's brain. According to some embodiments, aspects of the workflow 900 may be embodied in computer code stored in non-transitory computer readable storage. The workflow may be executed using computing resources, such as the clinician programmer 70 (FIG. 5), for example.

An aspect of the workflow 900 is algorithms that help the clinician select stimulation parameters without the clinician having to try an inordinate number of trial stimulations having different parameter settings. At step 902, the clinician uses the system to issue one or more trial stimulation waveforms using the implanted electrodes. The trial stimulation may also be referred to herein as “active stimulation,” or “test stimulation” to distinguish it from modeled stimulation, which is discussed below. According to some embodiments, the clinician may try one or a few trial stimulation waveforms. According to some embodiments, the algorithm(s) embodying the workflow may comprise macros or scripts that are configured to automatically run a plurality of trial stimulation waveforms having different parameter configurations.

At step 904, evoked potentials are recorded at one or more of the electrodes of the electrode lead(s). According to some embodiments, stimulation may be provided at one of the electrode contacts and the EPs may be sensed at the same contact. According to other embodiments, stimulation may be provided at one of the electrode contacts and the EPs may be sensed at each of the other contacts.

At step 906, features may be extracted from the recorded EPs from each of the electrodes. Examples of such features of the evoked potentials include but are not limited to:
a height of any peak (e.g., N1);
a peak-to-peak height between any two peaks (such as from N1 to P2);
a ratio of peak heights (e.g., N1/P2);
a peak width of any peak (e.g., the full-width half-maximum of N1);
an area or energy under any peak;
a total area or energy comprising the area or energy under positive peaks with the area or energy under negative peaks subtracted or added;
other measures of energy of magnitude of a peak or peaks, such as an RMS measure;
a length of any portion of the curve of the evoked potential (e.g., the length of the curve from P1 to N2, calculated by various methods, including piecewise sum);
any time defining the duration of at least a portion of the evoked potential (e.g., the time from P1 to N2);
latencies of any peaks (P1 . . . Pn, N1 . . . Nn, etc.) as well as other feature-to-feature latencies;
amplitude decay function;
a time delay from stimulation to issuance of the evoked potential, which is indicative of the neural conduction speed of the evoked potential, which can be different in different types of neural tissues, such delays optionally calculated from rising edges, falling edges, or at pre-determined positions within a pulse width, such positions variable and programmable, and including determination based in whole or part from prior data from same or different patients, or computational models;
a conduction speed (i.e., conduction velocity) of the evoked potential, which can be determined by sensing the evoked potential as it moves past different sensing electrodes;
a measure of variation of any of the previous or other features, e.g., variance or standard deviation;
a rate of variation of any of the features, i.e., how such features change over time, either within the same evoked response or between stimulation pulses;
parameters fit to models of rates of changes of features, e.g., envelope, dwell-time, decay constant;
a power (or energy) determined in a specified frequency band (e.g., delta, alpha, beta, gamma, etc.) determined in a specified time window (for example, a time window that overlaps the neural response, the stimulation artifact, etc.);
spectral characteristics in the frequency domain (e.g., Fourier transform);
a cross-correlation or cross-coherence of the evoked potential shape with a target optimal shape;
a nonlinear transform applied to the signal, such as a neural network;
a number of peaks observed following a single pulse and/or during a quiescent period during delivery of pulses;
differences between peaks and/or peak features within the same evoked response,
fit with “dose-response” characteristics; and
any mathematical combination or function of these features.

At step 908, the extracted features of the recorded EPs are compared to features of modeled EPs. As mentioned above, embodiments of the models described in this disclosure are configured to predict EP signals that will be sensed at some or all of the electrodes for a given set of stimulation parameters based on the extent to which those stimulation parameters activate the target volume of the patient's brain. The comparing of the EPs can be done in the time domain, the frequency domain, and/or features derived from both. At step 910, the comparisons are used to make recommendations regarding the stimulation. For example, the algorithms may recommend particular stimulation parameters that are likely to be effective for treating the patient.

FIG. 10 illustrates aspects of EP modeling, as described herein. The EP modeling described in the disclosure may be performed by the clinician programmer (CP) or another computing device by executing instructions embodied in one or more non-transitory computer readable media. FIG. 10 illustrates a side view and a top view of aspects of the EP modeling. Images as depicted in FIG. 10 may be displayed as features of a GUI on the CP according to some embodiments.

The illustrated embodiment of the EP model 1000 comprises a lead model 1002, a target volume model 1004, and a SFM 1006. The lead model 1002 may comprise a model of the geometric and electrical behavior of the lead. It may be computed based on finite element modeling (FEM) of the lead. The target volume model 1004 models one or more target volumes within the patient's brain. For example, the target volume model may comprise the STN and or a portion of the STN, such as the dorsal region of the STN. The target volume model may be based on imaging data such as fusion of MRI and CT images using individualized segmentation software auto-segmentation algorithms, as is known in the art. Example algorithms are included in commercial segmentation software, such as Brainlab Elements™, Brainlab, Germany. The relevant 3-D models may be voxelized, that is, the three-dimensional structure may be divided into volume elements, i.e., voxels. The SFM 1006 comprises a model of the activation of neural elements that will occur under a given set of stimulation parameters (e.g., amplitude, pulse width, frequency, duty cycle, etc.). Methods for generating SFMs are known in the art and are available in products, such as Boston Scientific Corporation's Guide™ XT product (Boston Scientific Corporation, USA). The EP model 1000 is configured to determine an overlap region 1008 of the SFM 1006 generated with the modeled stimulation parameter set with the target volume 1004. In other words, the overlap region represents the neural elements within the target volume that are expected to be stimulated by the modeled stimulation.

The EP model 1000 is further configured to model/predict the electrical signals that will be sensed at various electrodes of the electrode lead based on the neural activation within the overlap region 1008. This model is based on the understanding that neural elements within the overlap region 1008 are responsible for the EPs. In other words, neural elements within the SFM 1006 but not within the target volume 1008 do not produce EPs. Likewise, neural elements within the target volume 1008 but not within the SFM 1006 do not produce EPs. Embodiments of the EP model involve representing the neural space within the overlap region as a stimulation space and applying a unit current to each of the points within the space. Membrane currents of neurons within the overlap region are simulated from biophysical (e.g., Hodgkin-Huxley) models of the neurons targeted by stimulation and/or imported as time-series or frequency-series templates based on a priori simulations or recordings. To convert the membrane currents into the voltage signal representing the EP at the sensing contact, membrane currents are represented as point sources within a target tissue volume, scaled according to their distance from the sensing electrode or stimulation site, and summed over all space. Embodiments use the reciprocity principle, which holds that a potential (V) measured at a first point due to a current source (I) at a second point is equal to the potential measured at the second point due to appoint electrode at the first point. The relationship between I and V is known as “transfer impedance” or “transimpedance” and can be treated as constant regardless of I's strength. The voltage an electrode induced by an EP can be modeled as:

$$V ( electrode ) = I ( source ) / 4 πσ r$$

Where I(source) is the current flowing within the brain tissue generated by neural activity (i.e., the aforementioned “membrane currents”), a is the electrical conductivity of the brain tissue, r is the distance between the source of the neural activity and the electrode. The ¼πσr term is referred to as the transimpedance term and can be calculated using finite element modeling (FEM).

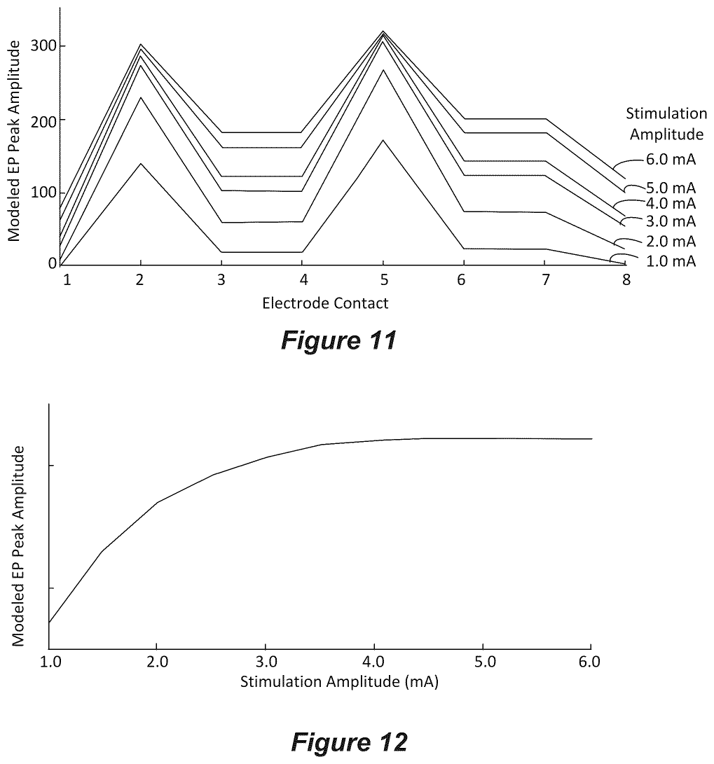

FIG. 11 illustrates normalized EP amplitudes calculated at different electrodes on the electrode lead for EPs evoked at different stimulation amplitudes. As expected, the amplitude of the modeled sensed EPs increase as the stimulation amplitude increases since the overlap of the SFM with the target volume would be expected to increase with increasing stimulation amplitude. The illustrated data show that stimulation at electrodes 2 and 5 result in the greatest overlap of the SFM and the target volume. FIG. 12 shows a graph of the modeled peak amplitude of the modeled sensed EPs as a function of stimulation amplitude. Notice that the modeled EP amplitude increases with increasing stimulation amplitude. This behavior is expected since greater stimulation amplitudes would be expected to result in SFMs with larger overlap regions with the target volume (i.e., more target neural elements activated). Also notice that the EP peak amplitude curve saturates a given stimulation amplitude. This behavior indicates that, at a certain point, the SFM completely overlaps the target volume and therefore activates all the available neural elements within the target volume. As a result, further increases in stimulation amplitude does not result in any further increase in the EP amplitude, and the observed plateau indicates the maximum sensed EP. Signals shown in FIGS. 11 and 12 are normalized to the maximum EP amplitude.

Embodiments of the EP modeling described herein allows a user to quickly interrogate a large number of stimulation programs, each having different parameter sets, and to predict the electrical signals (i.e., the EPs) that will be sensed at each of the electrodes resulting from stimulation using those programs. According to some embodiments, the modeling algorithm may predict the EPs that will be sensed at each of the electrodes for each of the stimulation programs. For example, the EP modeling algorithm may model stimulation at a first electrode and model the EP(s) that will be sensed at that first electrode. Alternatively, the algorithm may model stimulation at the first electrode and model EPs that will be sensed at each of the other electrodes. In either case, the model can predict which stimulation location activates the most of the target volume. Moreover, current steering and current fractionation can be used to model stimulation at locations that are between the electrodes. One or more of the EP features may be extracted for each of the sensed EPs.

The predicted EPs/EP features may be extracted stored, for example, in a look-up table (LUT). According to some embodiments, the predicted EPs/EP features may be used to suggest stimulation parameters that are likely to be effective for addressing the patient's symptoms and/or for minimizing side effects. For example, the saturation curve shown in FIG. 12 may be used to predict stimulation parameters that are likely to maximize the overlap of the stimulation field and the target volume without extending beyond the target volume, which might lead to side effects.

According to some embodiments, the clinician may use a small number of test stimulations with a patient and measure EPs evoked by those test stimulations. Those measured EPs can be compared to the predicted EPs contained in the LUT. The algorithm may use the comparison to suggest stimulation parameters that are likely to be effective. This saves the clinician from having to perform a large number of test stimulations to optimize the stimulation parameters. According to some embodiments, the clinician programmer (CP) or other computing device may be configured to execute a script that runs the limited number of test stimulations, compares the resulting EPs to the LUT, and suggests stimulation parameters. According to some embodiments, the algorithm may use machine learning and/or artificial intelligence (AI) to optimize the stimulation. For example, a machine learning algorithm may be trained on the predicted/measured EPs and use that training to predict the location of the electrode lead and suggest stimulation parameters. The machine learning algorithms can be updated based on clinical observations during the fitting process.

Implementations of the disclosed methods and systems may provide one or more graphical user interface (GUI) elements, for example, that may be implemented of the CP or other computing device. As mentioned above, a GUI may be configured to display a representation of the target volume, the SFM, and the overlap region, as shown in FIG. 10 for given stimulation parameters. According to some embodiments, a user may be able to define regions of interest (ROIs) and adjust them using the GUI. The user may be able to define and adjust the target resolution for signal prediction. According to some embodiments, the GUI and system may be configured to display and compare the actual measured signals at the electrodes with the predicted signals. For example, the signals may be overlayed. The user may be able to check the progression of the sensed EPs with stimulation amplitude to predict the relative position of the electrodes with respect to the target volume or source of the EP within the target volume. The user may be able to define EP features of interest, such as various peak or peak-peak amplitudes, area under the curve, confidence levels, etc. According to some embodiments, the user may be able to identify and flag extraneous signals that occur when stimulation is applied outside the target volume.

According to some embodiments, the disclosed methods and systems may be used to back-calculate the location of the source of the EPs based on EP amplitudes sensed during the stimulation sequence based on the modeled EPs using the LUT and/or machine learning algorithms. Such predictions can be compared to the imaging data described above, for example, the voxelized imaging data. According to some embodiments, the predicted locations may be displayed as a probability cloud, color or heat map, etc. According to some embodiments, the machine learning algorithms can be employed to predict or infer the EP signals on contacts that have missed recording signals.

According to some embodiments, the GUI may provide a representation of uncertainty in the suggested stimulation parameters. For example, if the clinician only uses a very few trial stimulations, then the model may be underdetermined, that is, there may be multiple predicted stimulation settings in the LUT that matches with the clinically determined measured EPs. Such uncertainty may be reflected in the GUI. According to some embodiment, the GUI may associate weights or probabilities with the suggested stimulation parameters. According to some embodiments, further measurements may be recommended. According to some embodiments, the uncertainty may be reduced and the models may be refined by incorporating further information, such as electrode-tissue interface (ETI) and device filtering, noise reduction, historic clinical data, and the like. According to some embodiments, EPs may be differentiated from an artifact or electrical noise based on one or more elements (or combinations thereof): features, morphology, amplitude relative to floor noise, and/or progression (or lack thereof) versus stimulation parameter (the AI and/or ML algorithm may be trained to do this as well).

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method of providing deep brain stimulation (DBS) to a patient's brain using one or more electrode leads implanted in the patient's brain, wherein each of the one or more leads comprises a plurality of electrodes, the method comprising:
using a first one or more of the electrodes to provide active stimulation to the patient's brain,
using a second one or more of the electrodes to record one or more electrical signals indicative of evoked potentials (EPs) evoked by a target volume of the patient's brain,
comparing the one or more recorded signals to a plurality of modeled EPs, wherein each of the modeled EPs comprise predicted electrical signals at one or more of the electrodes in response to activation of the target volume by modeled stimulation with a predefined set of model stimulation parameters, and
using the comparison to adjust the stimulation.

2. The method of claim 1, wherein comparing the one or more recorded signals to the modeled EPs comprises extracting one or more features of the recorded signals and comparing the extracted features to corresponding features of the modeled EPs.

3. The method of claim 2, wherein the one or more extracted features comprise one or more of one or more peak heights, one or more peak-peak heights, areas under one or more peaks, one or more latencies, and one or more peak-peak ratios.

4. The method of claim 1, wherein comparing the one or more recorded signals to the modeled EPs comprises overlaying the recorded signals and the modeled EPs.

5. The method of claim 1, wherein the modeled EPs are comprised within a look-up table (LUT).

6. The method of claim 1, wherein adjusting the stimulation comprises adjusting the stimulation to provide stimulation to a larger portion of the target volume.

7. The method of claim 1, wherein adjusting the stimulation comprises adjusting one or more of an amplitude, pulse width, frequency, duty cycle, electrode position, or stimulation location.

8. The method of claim 1, wherein adjusting the stimulation comprises adjusting which electrodes are active for providing stimulation and/or adjusting a fractionation of current among the electrodes that are active.

9. The method of claim 1, further comprising determining the modeled EPs for each of the predefined sets of model stimulation parameters.

10. The method of claim 9, wherein determining the modeled EPs comprises:
for each of the predefined sets of model stimulation parameters, determining a stimulation field model (SFM) that predicts a volume of tissue activated using the set of model stimulation parameters,
determining an overlap region of the SFM with the target volume, and
predicting electrical signals that will be sensed at the one or more of the electrodes based on activation of neural elements within the overlap region,
and wherein the method further comprises comparing the one or more recorded signals to the predicted electrical signals to predict an overlap of stimulation fields created by the active stimulation with the target volume.

11. A system for providing deep brain stimulation (DBS) to a patient's brain using one or more electrode leads implanted in the patient's brain, wherein each of the one or more leads comprises a plurality of electrodes, the system comprising:
control circuitry configured to execute a method comprising:
using a first one or more of the electrodes to provide active stimulation to the patient's brain,
using a second one or more of the electrodes to record one or more electrical signals indicative of evoked potentials (EPs) evoked by a target volume of the patient's brain,
comparing the one or more recorded signals to a plurality of modeled EPs, wherein each of the modeled EPs comprise predicted electrical signals at one or more of the electrodes in response to activation of the target volume by modeled stimulation with a predefined set of model stimulation parameters, and
using the comparison to adjust the stimulation.

12. The system of claim 11, wherein comparing the one or more recorded signal to the modeled EPs comprises extracting one or more features of the recorded signals and comparing the extracted features to corresponding features of the modeled EPs.

13. The system of claim 12, wherein the one or more extracted features comprise one or more of one or more peak heights, one or more peak-peak heights, areas under one or more peaks, one or more latencies, and one or more peak-peak ratios.

14. The system of claim 11, wherein comparing the one or more recorded signal to the modeled EPs comprises overlaying the recorded signals and the modeled EPs.

15. The system of claim 11, wherein the modeled EPs are comprised within a look-up table (LUT).

16. The system of claim 11, wherein adjusting the stimulation comprises adjusting the stimulation to provide stimulation to a larger portion of the target volume.

17. The system of claim 11, wherein adjusting the stimulation comprises adjusting one or more of an amplitude, pulse width, frequency, duty cycle, or stimulation location.

18. The system of claim 11, wherein adjusting the stimulation comprises adjusting which electrodes are active for providing stimulation and/or adjusting a fractionation of current among the electrodes that are active.

19. The system of claim 11, wherein the method further comprises determining the modeled EPs for each of the predefined sets of model stimulation parameters.

20. The system of claim 9, wherein determining the modeled EPs comprises:
for each of the predefined sets of model stimulation parameters, determining a stimulation field model (SFM) that predicts a volume of tissue activated using the set of model stimulation parameters,
determining an overlap region of the SFM with the target volume, and
predicting electrical signals that will be sensed at the one or more of the electrodes based on activation of neural elements within the overlap region.
